SEQUENCE LISTING (3 sequences)

SEQ ID NO: 1 - DNA, 26 bp, synthetic construct
  source 1..26: mol_type = other DNA
acactagccatccttactgcgcttcg

SEQ ID NO: 2 - DNA, 23 bp, synthetic construct
  source 1..23: mol_type = other DNA
accccgcattacgttggtggacc

SEQ ID NO: 3 - DNA, 20 bp, synthetic construct
  source 1..20: mol_type = other DNA
gaccccaaaatcagcgaaat

(19) United States
(12) Patent Application Publication
(10) Pub. No.: US 2025/0052752 A1
(43) Pub. Date: Feb. 13, 2025

(54) Graphene-Based Sensor for Detecting SARS-COV-2 Virus in a Biological Sample

(71) Applicant: Graphene-Dx, Inc., Boston, MA (US)

(72) Inventors: Namal NAWANA, Weston, MA (US); Reza MOLLAAGHABABA, Natick, MA (US); Mehdi ABEDI, Brighton, MA (US); Edward Alvin GREENFIELD, Stoughton, MA (US); Nirva PATEL, Weston, MA (US); Mohammed FOTOUHI, Boston, MA (US)

(73) Assignee: Graphene-Dx, Inc., Boston, MA (US)

(21) Appl. No.: 18/802,485

(22) Filed: Aug. 13, 2024

Related U.S. Application Data
(63) Continuation of application No. 17/208,692, filed on Mar. 22, 2021, now abandoned.
(60) Provisional application No. 63/023,014, filed on May 11, 2020.
(60) Provisional application No. 63/009,209, filed on Apr. 13, 2020.
(60) Provisional application No. 62/992,677, filed on Mar. 20, 2020.

(51) Int. Cl. G01N 33/569; G01N 27/12

(52) U.S. Cl. CPC G01N 33/56983; G01N 27/125; G01N 2333/165

(57) ABSTRACT

In one aspect, a sensor for detecting SARS-COV-2 virus in a sample, e.g., a blood sample, is disclosed, which includes a graphene layer, a plurality of binding agents coupled to said graphene layer to generate a functionalized graphene layer, where the binding agents exhibit specific binding to at least one epitope of SARS-COV-2 virus, and a plurality of electrical conductors electrically coupled to said functionalized graphene layer for measuring an electrical property (e.g., DC electrical resistance) of the functionalized graphene layer. While in some embodiments such binding agents are monoclonal antibodies, in other embodiments they can be polyclonal antibodies.

### CROSS-REFERENCE TO RELATED APPLICATIONS

The present application is a continuation application of U.S. application Ser. No. 17/208,692, filed on Mar. 22, 2021, which claims priority to U.S. Provisional Application Nos. 63/023,014: 63/009,209; and 62/992,677 filed on May 11, 2020; Apr. 13, 2020, and Mar. 20, 2020, respectively. The aforementioned applications are incorporated herein by reference in their entireties.

### REFERENCE TO AN ELECTRONIC SEQUENCE LISTING

The contents of the electronic sequence listing (50240017US02sequencelisting.xml; Size: 4 kb; and Date of Creation: Oct. 24, 2024) is herein incorporated by reference in its entirety.

### BACKGROUND

The present disclosure relates generally to a sensor and a method of using the sensor for detecting SARS-COV-2 virus in a biological sample, and more particularly to a Point-of-Care (POC) system for detecting SARS-COV-2 virus in a sample, such as, a nasopharyngeal sample, obtained from an individual.

Coronaviruses are enveloped non-segmented positive-sense RNA viruses that belong to the family Coronaviridae and can infect animals as well as humans. The severe acute respiratory syndrome coronavirus (SARS-COV) and Middle East respiratory syndrome coronavirus (MERS-CoV) have infected thousands of individuals in the past two decades with a mortality rate of 10% and 37%, respectively.

The recent emergence of a novel coronavirus (SARS-COV-2) has caused great health and economic uncertainties. The rapid transmission of the virus to many countries across the world has resulted in the World Health Organization (WHO) declaring a global pandemic. The early detection of an infection by SARS-COV-2 can be useful in inhibiting, and at least slowing down, the spread of the viral infection.

Accordingly, there is a need for device and methods for the detection of SARS-COV-2 virus and particularly for such devices that can be used at the point-of-care.

### SUMMARY

In one aspect, a sensor for detecting SARS-COV-2 in a biological sample, e.g., a nasopharyngeal sample, is disclosed, which comprises a graphene layer, a plurality of anti-SARS-COV-2 binding agents (e.g., antibodies and/or aptamers) coupled to the graphene layer to generate an antibody-functionalized graphene layer, and a plurality of electrical conductors electrically coupled to said functionalized graphene layer for measuring at least one electrical property of said functionalized graphene layer. The anti-SARS-COV-2 binding agents comprise antibodies or aptamers exhibiting specific binding to SARS-COV-2.

In some embodiments, the sensor can include a reference electrode for applying a reference AC signal, e.g., an AC voltage (herein also referred to as “AC signal”), and in some embodiments as well as a DC offset voltage (e.g., a DC ramp voltage, which is herein also referred to as a “DC signal”) to the functionalized graphene layer. By way of example, the reference AC signal can have a frequency in a range of about 1 kHz to about 1 MHZ, such as in a range of about 10 kHz to about 100 kHz, or in a range of about 50 kHz to about 200 kHz, or in a range of about 200 kHz to about 300 kHz, or in a range of about 400 kHz to about 700 kHz, and the amplitude of the applied AC voltage (e.g., the peak-to-peak amplitude) can be, for example, in a range of about 100 millivolts to about 3 volts, e.g., in a range of about 1 volt to about 2 volts.

As noted above, in some embodiments, a DC ramp voltage is applied to the reference electrode, together with the AC voltage, during data acquisition. The DC ramp voltage can vary, for example, from about-10 volts to about 10 volts, e.g., in a range of about-5 volts to about +5 volts, or in a range of about-3 volts to about +3 volts, or in a range of about-1 volt to about +1 volt.

In many embodiments, the sample includes a biological sample, such as a nasopharyngeal sample, a blood sample, a nasal secretion sample, or a throat secretion sample.

In some embodiments, the anti-SARS-COV-2 antibodies and/or aptamers are coupled to the graphene layer via a plurality of linkers. Each of the linkers is coupled at one end thereof, e.g., via a n-n bond, to the graphene layer and at another end, e.g., via a covalent bond, to at least one epitope of SARS-COV-2 virus. In some embodiments, the linkers include 1-pyrenebutonic acid succinimidyl ester.

In some embodiments, the graphene layer can be functionalized with a plurality of hydroxyl groups. In some such embodiments, the anti-SARS-COV-2 antibodies and/or aptamers are coupled to the hydroxyl groups via a plurality of aldehyde moieties.

In a related aspect, a method of detecting SARS-COV-2 virus in a biological sample, e.g., nasal swab, nasopharyngeal swab, oropharyngeal swab, saliva, whole blood, fingerstick, serum, and plasma.

In some embodiments, the at least one electrical property of the functionalized graphene layer that is measured in response to the exposure of the antibody and/or aptamer-functionalized graphene layer to a sample under test is a DC electrical resistance of the antibody and/or aptamer-functionalized graphene layer. For example, a change in the electrical resistance of the antibody and/or aptamer-functionalized graphene layer can be measured to determine whether SARS-COV-2 virus above the detection limit of the sensor is present in the sample.

In a related aspect, a method of fabricating a sensor for detecting SARS-COV-2 virus in a biological sample, e.g., a nasopharyngeal sample, is disclosed, which includes coupling a plurality of linkers, e.g., via n-n bonds, to a graphene layer deposited on an underlying substrate, and covalently coupling a plurality of antibodies and/or aptamers exhibiting specific binding to SARS-COV-2 virus to said linkers.

In a related aspect, a disposable cartridge for detecting SARS-COV-2 virus in a biological sample, e.g., a nasopharyngeal sample, is disclosed, which includes a microfluidic component having an inlet port for receiving a sample and an exit port. A sensor is fluidically coupled to the microfluidic component to receive at least a portion of the sample from the exit port. The sensor can include a graphene layer, a plurality of anti-SARS-COV-2 antibodies and/or aptamers coupled to the graphene layer to generate a functionalized graphene layer, and a plurality of electrical conductors electrically coupled to the antibody and/or aptamer-functionalized graphene layer for measuring an electrical property of said functionalized graphene layer.

In a related aspect, a graphene-based sensor is disclosed, which includes a graphene layer disposed on an underlying substrate, e.g., a semiconductor or a plastic substrate, and a protein, e.g., nucleocapsid or spike protein of SARS-COV-2 virus or fragments thereof, that is coupled to the underlying graphene layer and can bind to antibodies, such as IgG, IgM, and/or IgA antibodies, that are produced by an individual in response to infection by the SARS-COV-2 virus. At least a pair of electrically conductive electrodes are coupled to the antibody-functionalized graphene layer, e.g., they can be deposited on a portion of the graphene layer not functionalized with the proteins, so as to allow the measurement of at least one electrical property of the antibody-functionalized graphene layer, e.g., its DC electrical resistance, in response to exposure of the antibody-functionalized graphene layer to a sample. The anti-SARS-COV-2 antibodies in a sample, e.g., a serum sample or a saliva sample, obtained from an individual can be detected via observing an expected change in the electrical property of the antibody-functionalized graphene layer.

In some embodiments, a sensor according to the present teachings can include a graphene layer that is functionalized with a catalytically inactive CRISPR complex with a sgRNA having a oligonucleotide sequence that is complementary with a DNA sequence of interest. If the DNA sequence (e.g., a DNA sequence having a target mutation) is present in a sample (e.g., a nasopharyngeal sample), the binding of that DNA sequence to the sgRNA can cause a change in at least one electrical property of the functionalized graphene layer, which can be measured and analyzed as discussed below to identify the presence of the mutation in the genome.

In some embodiments, the S and/or the N protein can be coupled to the underlying graphene layer using a plurality of linkers, such as the linkers disclosed above.

In some embodiments, the microfluidic component is formed of a polymeric material, such as PDMS (poly-di-methyl-siloxane) and/or PMMA (poly methyl methacrylate).

Further understanding of various aspects of the present teachings can be obtained with reference to the following detailed description in conjunction with the associated drawings, which are described briefly below.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in this specification and constitute a part of it, illustrate several embodiments consistent with the disclosure. Together with the description, the drawings serve to explain the principles of the disclosure.

FIG. 1A schematically depicts a disposable cartridge according to an embodiment for detecting SARS-COV-2 virus in a sample,

FIG. 1B schematically depicts a graphene-based sensor employed in the cartridge depicted in FIG. 1A,

FIG. 2 is a schematic view of a graphene-based sensor according to an embodiment including a plurality of metallic pads for measuring an electrical property thereof in response to interaction with a sample under study,

FIG. 3A depicts a circuit diagram of an example of a voltage-measuring device that can be employed for measuring a voltage induced across an antibody-functionalized graphene layer in response to application of a current thereto,

FIG. 3B schematically depicts an analyzer in communication with the voltage-measuring device shown in FIG. 3A for receiving the voltage measured by the voltage-measuring device as well as the current applied to the antibody-functionalized graphene layer,

FIG. 3C depicts an example of implementation of the analyzer shown in FIG. 3A,

FIG. 3D schematically depicts an embodiment of the present teachings in which a signal generated by a functionalized graphene layer in response to application of an AC input signal is detected via a lock-in amplifier,

FIGS. 4A and 4B schematically depict a sensor according to an embodiment, which includes a AC reference electrode,

FIG. 4C schematically depicts a sensor according to an embodiment, which includes an AC reference electrode on the substrate,

FIG. 4D schematically depicts a combination of a ramp voltage and an AC voltage applied to the reference electrode of a sensor according to an embodiment of the present teachings,

FIG. 5 schematically depicts an array of graphene-based sensor in accordance with an embodiment,

FIG. 6 is a schematic partial view of the one of the sensing elements depicting the coupling of viral proteins via linkers to the underlying graphene layer,

FIG. 7 schematically depicts a hydroxyl-functionalized graphene layer,

FIG. 8 schematically depicts a hydroxyl-functionalized graphene layer to which antibodies are attached,

FIG. 9A schematically depicts a serpentine microfluidic channel that can be employed in some embodiments of a sensor according to the present teachings to cause passive mixing of a sample passing therethrough,

FIG. 9B schematically depicts a spiral microfluidic channel that can be employed in some embodiments of a sensor according to the present teachings to cause passive mixing of a sample passing therethrough,

FIG. 10 schematically depicts a sensor according to an embodiment in which a microfluidic channel in which active mixing elements are incorporated guides a sample from an inlet port to a graphene-based sensing element according to the present teachings,

FIG. 11 schematically depicts a sensor according to the present teachings having a plurality of sensing elements,

FIG. 12 schematically depicts a sensor according to an embodiment of the present teachings, which include a graphene layer functionalized with a plurality of oligonucleotides for detection of a target nucleotide sequence of SARS-COV-2 virus,

FIG. 13 schematically depicts a sensor according to an embodiment having a plurality of graphene-based sensing elements, and

FIG. 14 shows experimental results comparing conductivity of graphene layers functionalized with anti-spike protein antibodies and isotype control antibodies when exposed to a sample containing spike proteins.

### DETAILED DESCRIPTION

The present disclosure relates generally to a graphene-based sensor that can be employed for detecting SARS-COV-2 virus and/or antibodies generated in response to infection by SARS-CoV-2 virus or vaccination in a sample, such as a nasal swab, nasopharyngeal swab, oropharyngeal swab, saliva, whole blood, fingerstick, serum, and plasma. Various terms are used herein in accordance with their ordinary meanings in the art. The term “about” as used herein denotes a variation of at most 5%, 10%, 15%, or 20% around a numerical value. The term “detection limit” as used herein refers to a minimum concentration of SARS-COV-2 virus or anti-SARS-COV-2 antibodies in a sample that can be positively detected using a sensor according to the present teachings.

In one aspect, the present disclosure provides teachings that allow the detection of SARS-CoV-2 virus or anti-SARS-COV-2 antibodies in a sample under investigation via the binding of the viruses or the antibodies to a binding agent that is coupled to a graphene layer to generate a functionalized graphene layer and measuring a change in at least one electrical property of the functionalized graphene layer. Some example of such binding agents include, without limitation, an aptamer, an antibody, an antibody fragment, etc. In the following description, for ease of explanation, the term “antibody” is intended to refer to any suitable binding agent, i.e., any 25 binding agent that exhibits specific binding to SARS-COV-2 virus.

The term “antibody,” as used herein, may refer to a polypeptide that exhibit specific binding affinity, e.g., an immunoglobulin chain or fragment thereof, comprising at least one functional immunoglobulin variable domain sequence. An antibody encompasses full length antibodies and antibody fragments. In some embodiments, an antibody comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody.

The term “antibody” also encompasses whole or antigen binding fragments of domain, or signal domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

The term “aptamer,” as used herein, refers to an oligonucleotide or a peptide molecule that exhibits specific binding to a target molecule. Aptamers are typically created by selecting them from a large random pool of oligonucleotide or peptide sequences, but natural aptamer do also exist.

The term “oligonucleotide binding element” as used herein refers to any of a protein, a peptide and/or an oligonucleotide that exhibits specific binding to a target oligonucleotide, such as an RNA or a single strand DNA segment.

The term “electrical property” as used herein may include electron mobility, electrical impedance (e.g., DC or AC electrical resistance or both), and/or electrical capacitance.

As noted above, coronaviruses are enveloped non-segmented positive-sense RNA viruses that belong to the family Coronaviridiae. The emergence of a series of pneumonia cases of unknown cause in Wuhan, Hubei, China with clinical presentations that resembled viral pneumonia has led to the identification of a novel coronavirus, via deep sequencing analysis of lower respiratory tract sample, which has been named 2019 novel coronavirus (2019-nCOV, herein also referred to as SARS-COV-2 virus). The respiratory syndrome caused by the SARS-CoV-2 virus infection is commonly referred to as COVID-19.

SARS-COV-2 belongs to the Betacoronaviurs genus and has a genome size of about 30 kilobases, which encodes for multiple structural and non-structural proteins. The structural proteins include the spike(S) protein, the envelope (E) protein, the membrane (M) protein, and the nucleocapsid (N) protein.

In one aspect, the present teachings provide a graphene-based sensor having an antibody-functionalized graphene layer configured to exhibit specific binding to SARS-COV-2 virus present in a sample under study, such as a nasopharyngeal sample. As discussed below, monoclonal and polyclonal antibodies exhibiting specific binding to at least one epitope of at least one protein of the SARS-COV-2 virus, such as the S or N protein, can be employed.

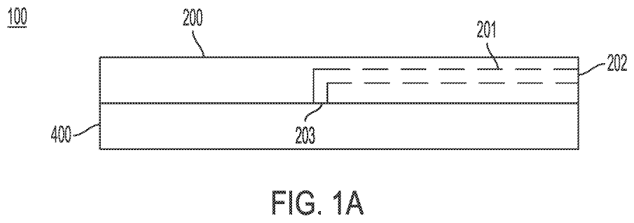

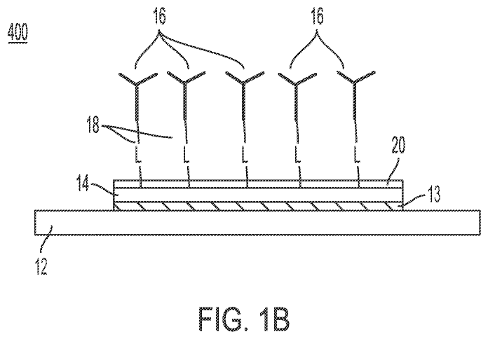

FIG. 1A schematically depicts cartridge 100 (herein also referred to as a cassette) according to an embodiment that can be employed to detect SARS-COV-2 virus in a sample, e.g., a naso-pharyngeal sample. In many embodiments, the cartridge 100 is a single-use and disposable cartridge.

The cartridge 100 includes a microfluidic delivery component 200 for delivering a sample under investigation to a sensor 400. In this embodiment, the microfluidic delivery component 200 includes at least one fluidic channel 201 that extends from an inlet port 202 through which a sample can be introduced into the microfluidic component to an outlet port 203 via which the sample can be delivered to the sensor 400. In some embodiments, the microfluidic channel can function based on capillary action. In some embodiments, the microfluidic delivery component 200 can be formed of a polymeric material, such as PDMS (polydimethylsiloxane) or PMMA (polymethyl methacrylate), and the microfluidic channel can be formed via etching or other known techniques in the art.

As shown schematically in FIGS. 1B and 1C, in this embodiment, the sensor 400 includes a graphene layer 14 that is disposed on an underlying substrate 12. While in some embodiments the substrate can be a semiconductor, in other embodiments, it can be a polymeric substrate. By way of example, in some embodiments, the substrate can be a silicon substrate while in other embodiments it can be a plastic substrate. For example, the underlying substrate can be formed of PDMS. Yet, in other embodiments, the underlying substrate can be a metallic substrate, such as a copper substrate. In this embodiment, the substrate 12 is a silicon substrate and a silicon oxide layer 13 separates the substate 12 from the graphene layer.

In this embodiment, the graphene layer is functionalized with a plurality of antibodies 16 that exhibit specific binding to at least one epitope of the SARS-COV-2 virus. The antibodies 16 can be monoclonal or polyclonal antibodies. An example of a method for generating monoclonal antibodies exhibiting specific binding to SARS-COV-2 virus is described below. Further, some examples of commercially available anti-SARS-COV-2 antibodies include, without limitation:

### Anti-N for SARS-COV-2:

mouse mAb from Genetex (GTX632269)
mouse mAb from Ray Biotech (128-10166-1)
mouse mAb from MyBiosource (MBS569937)

### Anti-S for SARS-COV-2:

mouse mAb from Ray Biotech (128-10168-1)
rabbit pAb from Sino Biological (40150-T62)
rabbit pAb from Sino Biological (40150-R007)

As shown schematically in FIG. 1B, a variety of linker molecules 18 can be employed for coupling the anti-SARS-COV-2 antibodies to the underlying graphene layer. By way of example, in some embodiments. 1-pyrenebutonic acid succinimidyl ester is employed as a linker to facilitate the coupling of the anti-SARS-COV-2 antibodies to the underlying graphene layer. In this embodiment, the plurality of anti-SARS-COV-2 antibodies can cover a fraction of, or the entire, surface of the graphene layer. In various embodiments, the fraction can be at least about 60%, at least about 70%, at least about 80%, or 100% of the surface of the graphene layer. The remainder of the surface of the graphene layer (i.e., the surface areas not functionalized with the antibodies) can be passivated via a passivation layer 20. By way of example, the passivation layer can be formed by using Tween-20, BLOTTO, BSA (Bovine Serum Albumin), gelatin or 3 mM APA (amino-PEGS-alcohol).

The passivation layer can inhibit, and preferably prevent, the interaction of a sample of interest introduced onto the graphene layer with areas of the graphene layer that are not functionalized with the antibodies. This can in turn lower the noise in the electrical signals that will be generated as a result of the interaction of the analyte of interest with the antibody molecules.

By way of example, in some embodiments, a graphene layer formed on an underlying substrate (e.g., plastic, a semiconductor, such as silicon, or a metal substrate, such as a copper film) can be incubated with the linker molecules (e.g., a 5 mM solution of 1-pyrenebutonic acid succinimidyl ester) for a few hours (e.g., 2 hours) at room temperature.

The linker modified graphene layer can then be incubated with the antibody of interest in a buffer solution (e.g., NaCO3-NaHCO₃buffer solution (pH 9)) at a selected temperature and for a selected duration (e.g., 7-10 hours at 4 C), followed by rinsing with deionized (DI) water and phosphate buffered solution (PBS). In order to quench the unreacted succinimidyl ester groups. the modified graphene layer can be incubated with ethanolamine (e.g., 0.1 M solution at a pH of 9 for 1 hour).

Subsequently, the non-functionalized graphene areas can be passivated via a passivation layer, such the passivation layer 20, schematically depicted in FIG. 1B. By way of example, the passivation of the non-functionalized portions of the graphene layer can be achieved, e.g., via incubation with 0.1% Tween-20.

In some embodiments, the graphene layer can be functionalized with protein G (PrG), which can be coupled to the underlying graphene layer via n-n interaction. The antibodies can be then covalently attached to the PrG. In some embodiments, the PrG can advantageously orient the antibodies so as to enhance the detection of the SARS-COV-2 virus. For example, a graphene layer deposited on an underlying substrate can be incubated in a solution of PrG in DMF (dimethyl formamide) (e.g., 100 μg/ml) for a few hours (e.g., 2-10 hours). Further details regarding functionalizing a graphene layer with PrG can be found, e.g., in an article entitled “An investigation into non-covalent functionalization of a single-walled carbon nanotube and a graphene sheet with protein G: A combined experimental and molecular dynamics study,” published in Scientific Reports (2019) 9:1273, which is herein incorporated by reference in its entirety.

The PrG functionalized graphene layer can then be incubated with the antibody of interest in a buffer solution (e.g., NaCQ3-NaHCQ3 buffer solution (pH 9)) at a selected temperature and for a selected duration (e.g., 7-10 hours at 4 C), followed by rinsing with deionized (DI) water and phosphate buffered solution (PBS). In order to quench the unreacted succinimidyl ester groups, the modified graphene layer can be incubated with ethanolamine (e.g., 0.1 M solution at a pH of 9 for 1 hour). Although the use of PrG protein for coupling antibodies to an underlying graphene layer is discussed herein in connection with antibodies exhibiting specific binding to SARS-COV-2 virus, it should be understood that such PrG functionalized graphene layer can also be employed for coupling other types of antibodies, e.g., antibodies exhibiting specific binding to other pathogens, such as chlamydia, to a graphene layer.

In some embodiments, rather than functionalizing the graphene layer with antibodies that exhibit specific binding to at least one protein associated with the SARS-COV-2 virus, the graphene layer can be functionalized with one or more proteins associated with the SARS-COV-2 virus so as to detect antibodies generated by an infected person in a biological sample extracted from such a person. In such embodiments, the presence of the antibodies reactive against SARS-CoV-2 virus may indicate that the person is immune to COVID-19 as a result of prior infection or vaccination.

Two to three days post-infection by SARS-COV-2 virus, a person's immune system releases antibodies (IgM) subclass. As the infection develops, this response changes mainly to an IgG subclass response. These antibodies can bind to the viral proteins coupled to the graphene layer and cause a change in an electrical property of the graphene layer, e.g., its electrical resistance. By way of example, in some such embodiments, a graphene layer can be functionalized with N and/or S protein associated with SARS-COV-2 virus. Further, a saliva sample may contain IgA antibodies.

More particularly, a biological sample extracted from the patient, e.g., a blood or a saliva sample, can be brought into contact with the functionalized graphene layer and a change in an electrical property of the functionalized graphene layer, e.g., a change in its DC electrical resistance, can be measured, e.g., in a manner disclosed herein, to detect the presence of such antibodies in the biological sample.

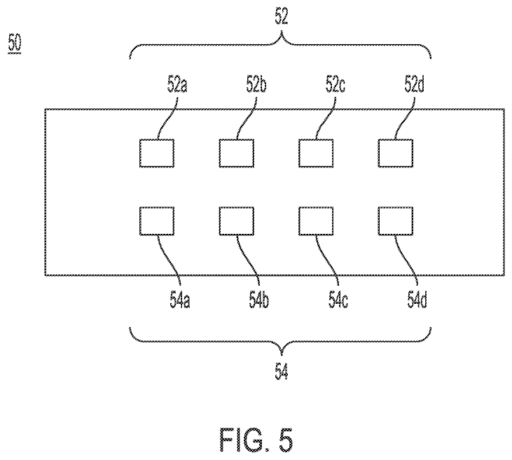

In some embodiments, the cartridge according to the present teachings can include an array of sensing elements (See, e.g., FIG. 5), where at least two of the sensing elements have graphene layers functionalized with different viral proteins. By way of example, one of the sensing elements can be functionalized with the N protein and the other with the S protein associated with SARS-COV-2 virus. In some such embodiments, the signals generated by such sensing elements can be averaged to generate a resultant signal, which can be analyzed for the 20 detection of SARS-COV-2 virus in a biological sample extracted from an individual suspected of having been infected by the SARS-COV-2 virus.

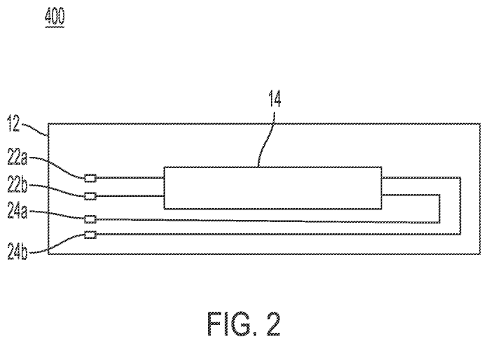

FIG. 2 shows a sensor 400 according to some embodiments, which includes electrically conductive pads 22a, 22b, 24a and 24b, that allow four point measurement of modulation of an electrical property of the functionalized graphene layer 14, e.g., its electrical resistance, in response to interaction of SARS-COV-2 virus present in a sample with the anti-SARS-COV-2 antibodies coupled to the graphene layer 14. In particular, in this embodiment, the conductive pads 22a/22b are disposed on the substrate 12 and electrically coupled to one end of the functionalized graphene layer 14 and the conductive pads 24a/24b are disposed on the substrate 12 and electrically coupled to the opposed end of the functionalized graphene layer 14 to allow measuring a change in an electrical property of the underlying graphene layer 14 caused by the interaction of SARS-COV-2 protein in a sample under study with the anti-SARS-COV-2 antibodies that are coupled to the graphene layer 14. By way of example, in this embodiment, a change in the DC resistance of the underlying graphene layer 14 can be monitored to determine the presence of SARS-COV-2 virus in a biological sample, such as a nasopharyngeal sample, a plasma sample, under study.

In other embodiments, a change in electrical impedance of the graphene layer 14 characterized by a combination of DC resistance and capacitance of the graphene/antibody system can be monitored to detect protein in a sample under study. The electrically conductive pads can be formed using a variety of metals, such as copper and copper alloys, among others.

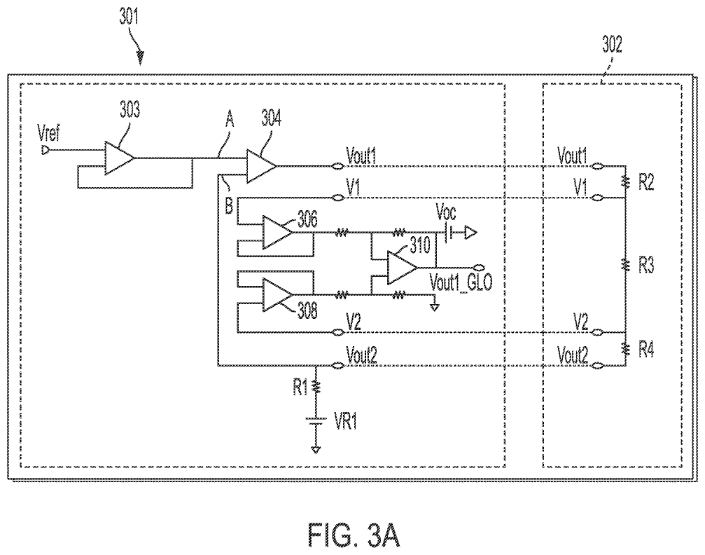

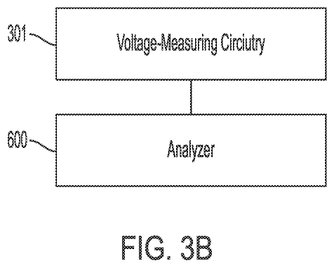

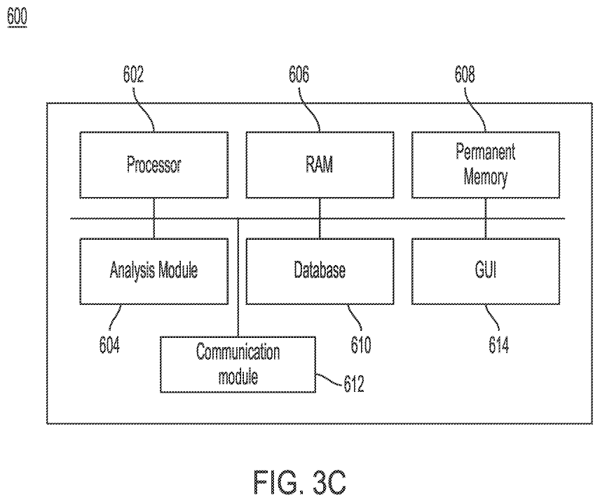

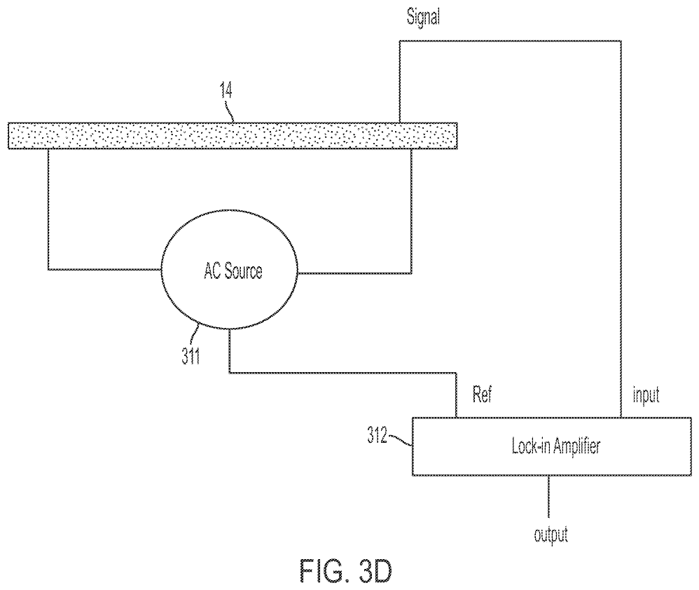

By way of example, FIG. 3A schematically depicts a voltage measuring circuitry 301 that can be employed in some embodiments of the present teachings. This figure shows a sensor 302 as an equivalent circuit corresponding to an antibody-functionalized graphene layer. A fixed voltage V (e.g., 1.2 V) is generated at the output of a buffer operational amplifier 303. This voltage is applied to one input (A) of a downstream operational amplifier 304 whose other input Bis coupled to VR1 ground via a resistor R1. The output of the operational amplifier 304 (Youn) is coupled to one end of the sensor 302 and the end of the resistor R1 that is not connected to VR1 ground is coupled to the other end of the sensor 302 (in this schematic diagram, resistor R2 denotes the resistance between two electrode pads at one end of the equivalent sensor 302, resistor R3 denotes the resistance of the graphene layer extending between two inner electrodes of the sensor, and resistor R4 denotes the resistance between two electrode pads at the other end of the sensor). As the operational amplifier maintains the voltage at the end of the resistor R1 that is not connected to VR1 ground at the fixed voltage applied to its input (A), e.g., 1.2 V, a constant current source is generated that provides a constant current flow through the sensor 302 and returns to ground via the resistor R1 and VR1.

The voltage generated across the antibody-functionalized graphene layer is measured via the two inner electrodes of the sensor. Specifically, one pair of the inner electrode pads is coupled to a buffer operational amplifier 306 and the other pair is coupled to the other buffer operational amplifier 308. The outputs of the buffer operational amplifiers are applied to the input ports of a differential amplifier 310 whose output port provides the voltage difference across the antibody-functionalized graphene layer. This voltage difference (Vout1-GLO) can then be used to measure the resistance exhibited by the antibody-functionalized graphene layer. The current forced through R3 is set by I=(Vref−VR1)/R1, where the value of VR1 is digitally controlled. For each value of current I, the corresponding voltage (Vout1_GLO) is measured and stored. The resistance of the antibody-functionalized graphene layer can be calculated as the derivative of the voltage, Vout1_GLO, with respect to current I, i.e., R=dV/d1.

As shown schematically in FIG. 3B, in some embodiments, an analyzer 600 can be in communication with the voltage measuring circuitry 301 to receive the applied current and the measured voltage value and use these values to calculate the resistance of the antibody-functionalized graphene layer. The analyzer 600 can then employ the calculated resistance, e.g., a change in the resistance in response to exposure of the antibody-functionalized graphene layer to a sample under investigation, to determine, in accordance with the present teachings, whether the sample contains SARS-COV-2 virus.

By way of example, as shown schematically in FIG. 3C, in this embodiment, the analyzer 600 can include a processor 602, an analysis module 604, a random access memory (RAM) 606, a permanent memory 608, a database 610, a communication module 612, and a graphical user interface (GUI) 614. The analyzer 600 can employ the communication module 612 to communicate with the voltage measuring circuitry 301 to receive the values of the applied current and the measured voltage. The communication module 612 can be a wired or a wireless communication module. The analyzer 600 further includes a graphical user interface (GUI) 614 that allows a user to interact with the analyzer 600.

By way of example, the analysis module 604 can employ the values of a current applied to the antibody-functionalized graphene layer as well as the voltage induced across the graphene layer to calculate a change in the resistance of the antibody-functionalized graphene layer in response to exposure thereof to a sample under investigation (e.g., using Ohm's law). The instructions for such calculation can be stored in the permanent memory 608 and can be transferred at runtime to RAM 606 via processor 602 for use by the analysis module 604. The GUI 614 can allow a user to interact with the analyzer 600.

In some embodiments, the analyzer 600 can include an AC (alternating current) source of current, which can apply an AC current having a known amplitude and frequency to the graphene layer. In particular, various embodiments can advantageously use an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 KHz to about 200 kHz. By way of example, the amplitude of the AC voltage applied to the reference electrode can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt.

The analyzer 600 can further include an ac voltmeter circuitry for measuring the ac voltage induced across the graphene layer in response to the application of the ac current to the layer. By measuring the amplitude and/or phase shift of the induced ac voltage, the electrical impedance of the graphene layer can be determined in a manner known in the art. In other embodiments, an AC voltage having a fixed frequency and amplitude can be applied to the functionalized graphene layer and the current can be monitored for detecting specific binding of SARS-COV-2 virus to the antibodies coupled to the graphene layer.

As shown schematically in FIG. 3D, in some embodiments, an AC source 311 can be utilized to apply an AC voltage or an AC current, e.g., with a frequency in a range of about 20 kHz to about 100 MHz, across, or through, an antibody-functionalized graphene layer, such as a graphene layer functionalized with antibodies exhibiting specific binding to SARS-COV-2 virus. The AC voltage or current can also provide a reference signal to a lock-in amplifier 312 whose input port receives a signal associated with the functionalized graphene layer in response to the application of AC voltage or current (e.g., AC voltage or current depending on the signal applied to the functionalized graphene layer). The output of the lock-in amplifier 312 can be used to determine whether an antigen of interest (e.g., SARS-COV-2 virus in this example) is present in a sample under study. For example, if the output of the lock-in amplifier 312 exceeds a predefined threshold, the presence of a target analyte (e.g., an anti-SARS-COV-2 virus or antibodies generated in response to viral infection) can be confirmed. Although the description of the lock-in detection is provided herein in connection with the detection of SARS-COV-2 virus, it should be understood that it can be employed for detecting other types of pathogens, such as Chlamydia bacterium.

Further details regarding a suitable analyzer that can be employed in the practice of some embodiments of the present teachings can be found, e.g., in U.S. Pat. No. 9,664,674 titled “Device and Method for Chemical Analysis,” which is herein incorporated by reference in its entirety.

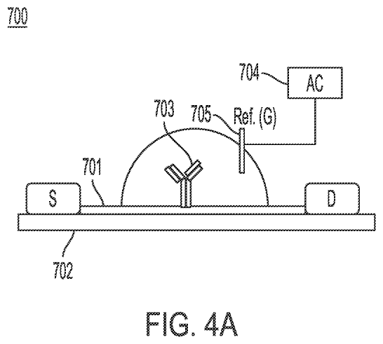

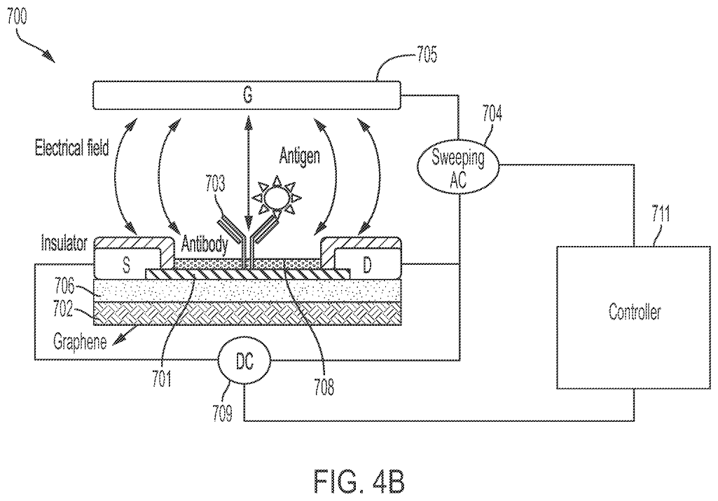

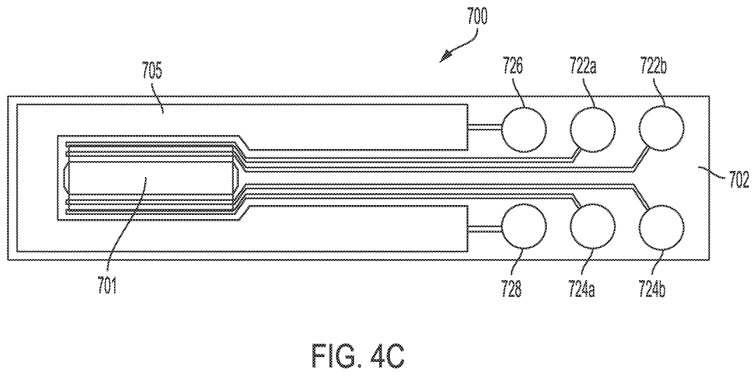

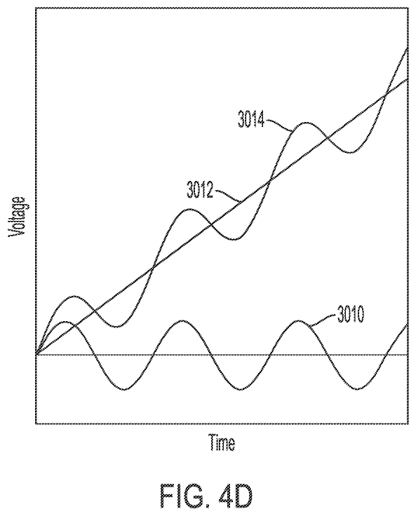

FIGS. 4A, 4B, and 4C schematically depicts another embodiment of a sensor 700 according to the present teachings. The sensor 700 includes a graphene layer 701 that is disposed on an underlying substrate 702, e.g., a semiconductor substrate, and is functionalized with an anti-SARS-COV-2 antibody 703. The remainder of the surface of the graphene layer 701 (i.e., the surface areas not functionalized with the antibodies) can be passivated via a passivation layer 708. In this embodiment, a silicon oxide layer 706 separates the graphene layer from the underlying substrate. A source electrode(S) and a drain electrode (D) are electrically coupled to the graphene layer to allow measuring a change in one or more electrical parameters of the functionalized graphene layer in response to interaction of the functionalized graphene layer with a sample.

Referring to FIG. 4C, for four point measurement of modulation of an electrical property of the functionalized graphene layer 701, the sensor 700 can include electrically conductive pads 722a, 722b, 724a, and 724b, similar to the embodiment shown in FIG. 2. The sensor 700 further includes a reference electrode (G) 705 that is disposed in proximity of the graphene layer. In some embodiments, the reference electrode (G) 705 is disposed on the same substrate 702 as that on which the graphene layer 701 is disposed (in other words, the reference electrode 705 is in substantially same plane as the graphene layer 701). In some embodiments, as shown in FIG. 4C, the reference electrode 705 can substantially surround the graphene layer 701. The reference electrode 705 can be electrically connected to additional conductive pads 726 and 728 to allow application of an AC voltage as well as a DC ramp voltage to the reference electrode 705, e.g., in a manner discussed above.

In use, in some embodiments, a change in the electrical resistance of the functionalized graphene layer can be measured in response to the interaction of the functionalized graphene layer with a sample, e.g., human serum, to detect SARS-COV-2 virus in a sample.

In some embodiments, the application of an AC (alternating current) reference voltage via an AC voltage source 704 to the graphene layer can facilitate the detection of one or more electrical properties of the functionalized graphene, e.g., a change in its resistance in response to the interaction of the antibody with an analyte exhibiting specific binding to the antibody. In particular, in some embodiments, the application of an AC voltage having a frequency in a range of about 1 kHz to 1 MHz, e.g., in a range of about 10 kHz to about 500 kHz or in a range of about 20 kHz to about 100 kHz, can be especially advantageous in this regard. By way of example, the amplitude of the AC voltage applied to the reference electrode can be in a range of about 1 millivolt to about 3 volts, e.g., 0.5 volts to 1 volt. Further, in some cases, the voltage applied to the reference electrode can have an AC component and a DC offset, where the DC offset can be in a range of about-40 volts to about +40 volts, e.g., −1 volt to about +1 volt.

By way of illustration, FIG. 4D schematically depicts a combination of an AC voltage 3010 and a DC offset voltage 3012 applied to the reference electrode, resulting in voltage 3014. By way of example, the DC offset voltage can extend from about-10 V to about 10 V (e.g., from −1 V to about 1 V), and the applied AC voltage can have the frequencies and amplitudes disclosed above.

Further, a DC source 709 can apply a DC voltage or current to the antibody-functionalized graphene layer, where a predefined change in an electrical response of the antibody-functionalized layer to the applied de voltage or current can indicate the present of a target analyte in a sample under study. A controller 711 can control the operation of the AC and DC sources. The controller can be implemented in hardware, software and/or firmware using techniques known in the art as informed by the present teachings. For example, the controller can be implemented in a manner discussed above in connection with FIG. 3C for the analyzer.

Without being limited to any particular theory, in some embodiments, it is expected that the application of such a voltage to the reference electrode can minimize, and preferably eliminate, an effective capacitance associated with a sample, e.g., such as human serum, with which the functionalized graphene layer is brought into contact as the sample is being tested, thereby facilitating the detection of a change in the resistance of the underlying graphene layer in response to the interaction of the antibodies 703 with a respective antigen. In some cases, the effective capacitance of the sample can be due to ions present in the sample.

The sensors and the methods of the present teachings can be employed to detect SARS-CoV-2 virus in a variety of samples, such as those disclosed above.

In some embodiments, a sensor according to the present teachings can include an array of sensing elements whose signals can be averaged to generate a resultant signal indicative of presence or absence of SARS-COV-2 virus (e.g., above a predefined threshold) in a sample, e.g., a plasma sample.

By way of example, FIG. 5 schematically depicts such a sensor 50 having a plurality of sensing elements 52a, 52b, 52c, and 52d (herein collectively referred to as sensing elements 52) and sensing elements 54a, 54b, 54c, and 54d (herein collectively referred to as sensing elements 54). Each of the sensing elements 52 and 54 includes a graphene layer functionalized with an anti-SARS-COV-2 antibody and has a structure similar to that discussed above in connection with sensor 400 or 700. In some embodiments, the different sensing elements can be functionalized with different types of anti-SARS-CoV-2 antibodies, e.g. antibodies exhibiting specific binding to different epitopes of the SARS-COV-2 virus. In some embodiments, the signals generated by the sensing elements 52 can be averaged to generate a resultant signal. Further, in some embodiments, at least one of the sensing elements 52 can be configured as a calibration sensing element to allow quantification of SARS-COV-2 virus in a sample, e.g., human serum. By way of example, the calibration can be achieved by utilizing a calibrated sample and detecting a change in at least one electrical property of the functionalized graphene layer, e.g., a change in its electrical in response to exposure to the calibration sample.

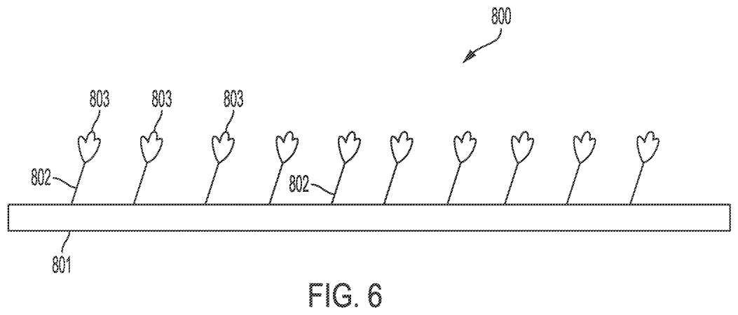

In some embodiments, instead of, or in addition to, functionalizing a graphene layer with anit-SARS-COV-2 antibodies, the graphene layer can be functionalized with one or more viral proteins, e.g., the N and/or S protein, to detect antibodies (e.g., antibodies reactive against SARS-COV-2 virus) generated by an individual in response to infection by the SARS-COV-2 or by vaccination. FIG. 6 is a partial schematic view of such a sensor 800 that includes a substrate 801 to which a plurality of S and/or N proteins 803 are coupled via a plurality of linkers 802, such as those disclosed herein.

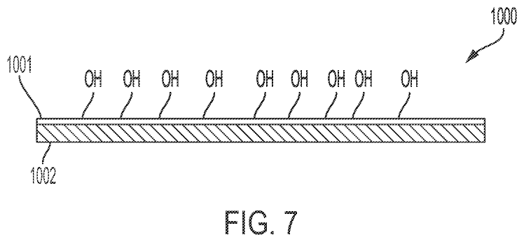

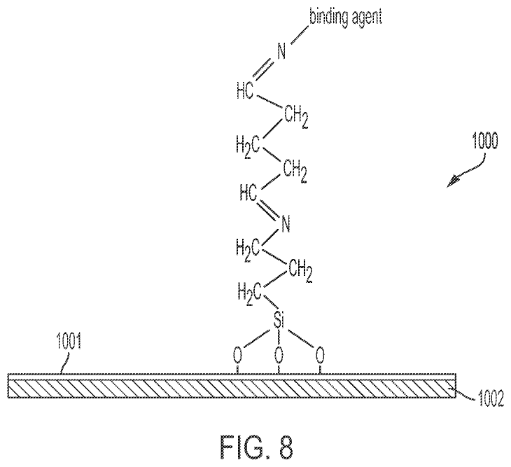

With reference to FIGS. 7 and 8, in some embodiments, the sensor 1000 can include a hydroxyl-functionalized graphene layer 1001 that is further functionalized with anti-SARS-COV-2 antibodies via a molecule containing an aldehyde moiety.

More specifically, with reference to FIG. 8, in this embodiment, the hydroxyl-functionalized graphene layer 1001 can be incubated with 2% 3-Aminopropyl triethoxysilane (APTES) in 95% ethanol for 1 hour to allow for aqueous silanization of the surface. The graphene layer can then be incubated in 2.5% glutaraldehyde in milli-Q water for a few hours (e.g., for 2 hours). This incubation can create aldehyde groups (—COH), which can react with amine groups (—NH2) of the antibody, e.g., via a covalent bond, thus coupling the antibody to the hydroxyl-functionalized graphene layer.

Similar to the previous embodiment, in this embodiment, the graphene layer 1001 can be initially deposited on an underlying substrate 1002. The underlying substrate 1002 can be, for example, a semiconductor, such as silicon, or a polymeric substrate, e.g., plastic.

Though not shown in FIG. 8, similar to the above sensor 700, the sensor includes metallic pads that can allow application of an electrical signal (e.g., a current or a voltage) to the antibody-functionalized graphene layer and monitor at least one electrical property of the antibody-functionalized graphene layer, e.g., its DC electrical resistance.

An advantage of a graphene-based sensor according to the present teachings is that it can be utilized at the point of sample collection by a lay person with minimal technical expertise. Another advantage of a graphene-based sensor according to the present teachings is that it can provide the detection results rapidly. For example, in some embodiments, a graphene-based sensor according to the present teaching can detect and quantify SARS-COV-2 in a sample, e.g., human serum, within a few minutes, e.g., 1-5 minutes including sample preparation steps.

As noted above, in some embodiments, a sensor according to the present teachings can include a microfluidic channel for guiding a sample, e.g., blood, from an inlet port, which receives a sample, to an outlet port through which the sample is delivered to a graphene-based sensing element according to the present teachings. In some such embodiments, such a microfluidic channel can include passive and/or active mixing elements for mixing the sample as the sample passes through the microfluidic channel.

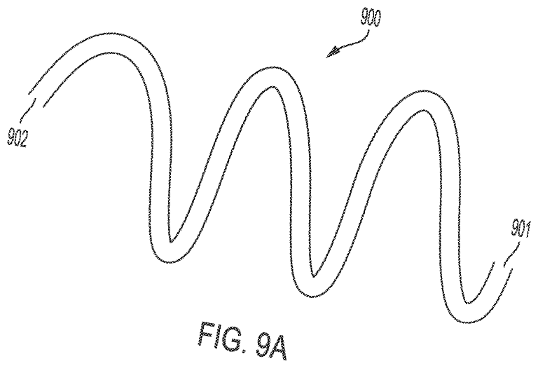

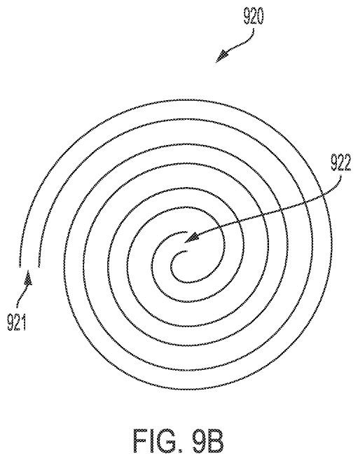

By way of example, FIG. 9A schematically depicts such a microfluidic channel 900 that has a serpentine shape extending from an inlet port 901 to an outlet port 902, where the serpentine shape of the microfluidic channel provides passive mixing of the sample as the sample passes through the channel. FIG. 9B shows another microfluidic channel 920 that has a spiral shape extending from an inlet port 921 to an outlet port 922, where the serpentine shape of the channel provides passive mixing of a sample passing through it. Other shapes, such as herringbone, can also be employed for a microfluidic channel that can provide mixing of a sample. Further, in some embodiments, obstacles can be provided in the microfluidic channel, instead of or in addition to configuring the shape of the channel, to provide mixing of a sample as it passes through the channel.

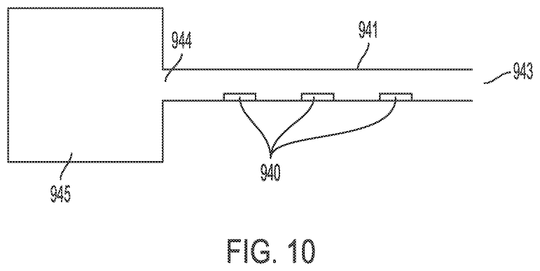

Yet, in other embodiments, a sensor according to the present teachings can include active mixing elements, micro-fluidic pumps, for mixing a sample, e.g., human serum. By way of example, as shown schematically in FIG. 10, in some other embodiments, one or more piezo electric elements 940 can be disposed in the microfluidic channel 941, which extends from an inlet port 943 to an outlet port 944, which delivers a sample to graphene-based sensing element 945 according to the present teachings. The piezoelectric elements 940 can be actuated to cause mixing of a sample as it passes through the microfluidic channel 941.

Regarding the epidemiology of SARS-COV virus, researchers studied the 2003 severe acute respiratory syndrome (SARS) outbreak and reported the temporal evolution of the virus titer after the onset of infection (Chen et al., “Cellular Immune Responses to Severe Acute Respiratory Syndrome Coronavirus (SARS-COV) Infection in Senescent BALB/c Mice: CD4+T Cells Are Important in Control of SARS-COV Infection,” J. Virol., 2010, 84 (3) 1289-1301: Hsueh et al., “Chronological evolution of IgM, IgA, IgG and neutralization antibodies after infection with SARS-associated coronavirus,” Clin. Microbial. Infect., 2004, 10 (12) 1062-1066; Glass et al., “Mechanisms of host defense following severe acute respiratory syndrome coronavirus pulmonary infection of mice,” J. Immunol., 2004, 173 (6) 4030-4039; Zhu, “SARS Immunity and Vaccination,” Cell. Mol. Immunol., 2004, 1 (3) 193-198). The peak of the virus titer occurs around 3-4 days after the infection, and then declines to reach very low values around day 8-9 after the onset of the infection. At this point, the infected individual begins to produce IgG and IgM antibodies in response to the infection with the IgG titer increasing more rapidly than the IgM titer

In some embodiments, a sensor according to the present teachings can include multiple graphene-based sensing elements, one of which is functionalized with an antibody that exhibits specific binding to at least one viral protein of the SARS-COV-2 virus, e.g., N and/or S protein of the SARS-COV-2 virus, another one of which is functionalized with the N viral protein of the SARS-COV-2 and the other is functionalized with the S viral protein of the SARS-COV-2 virus.

The sensing element that is configured to detect the SARS-COV-2 virus can be used to determine whether the SARS-COV-2 virus can be detected in a sample obtained via a nasal swab, e.g., a nasopharyngeal swab, from an individual. In some such embodiments, a phosphate buffer solution can be employed to extract SARS-COV-2 virus, if any, collected by a nasal swab employed to test an individual suspected of having been infected by SARS-COV-2 virus. Further, a blood sample obtained from such an individual can be introduced into the sensing elements that include a graphene layer functionalized with S and N proteins to determine the presence of IgG and/or IgM antibodies, if any, in the blood sample.

Further, in some embodiments, a sensor according to the present teachings can include a pair of graphene-based sensing elements functionalized with S protein, a pair of graphene-based sensing elements functionalized with N protein and one or more sensing elements functionalized with one or more antibodies that exhibit specific binding to one or more viral proteins. In such embodiments, for each pair of N and S-functionalized graphene-based sensing elements, one of the sensing elements of the pair can include a port configured for receiving a sample collected via a nasal swab and another port for receiving a blood sample (e.g., obtained via a finger prick) or a saliva sample. In this manner, one of the sensing elements of the pair can be employed to detect IgA antibodies, if any, present in the sample obtained via the nasal swab or a saliva sample that exhibit specific binding to the viral proteins coupled to the graphene layer and the other sensing element can be employed to detect any of IgG and/or IgM antibodies, if any, in a blood sample that exhibit specific binding to the viral proteins (e.g., Nor S protein) coupled to the graphene layer.

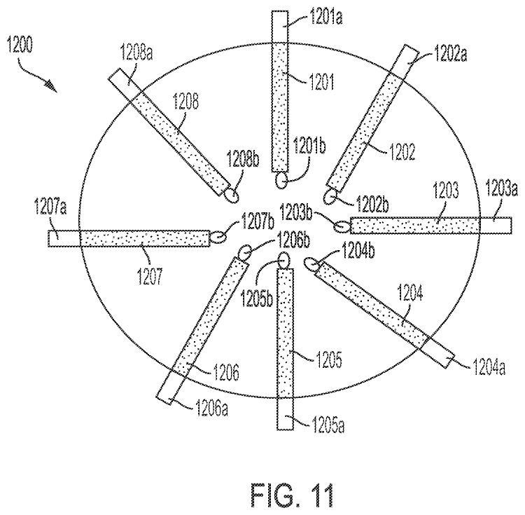

FIG. 11 schematically depicts a sensor 1200 according to an embodiment, which includes a plurality of graphene-based sensing elements 1201, 1202, 1203, 1204, 1205, 1206, 1207, and 1208 according to the present teachings. In this embodiment, the sensing element 1201 includes a graphene layer that is functionalized with an antibody that exhibits specific binding to the N protein of the SARS-COV-2 virus, and the sensing element 1205 includes a graphene layer that is functionalized with an antibody that exhibits specific binding to the S viral protein. These sensing elements include ports 1201b and 1205b, which are configured to receive a liquid sample generated by using a diluent (e.g., a phosphate buffer solution) to extract viruses, if any, from a nasal swab used to collect a mucosal sample, and a saliva sample, from an individual.

In this embodiment, each of the sensing elements 1202 and 1206 includes a graphene layer that is functionalized with the N viral protein. The sensing element 1202 includes a port 1202b configured for receiving a blood sample, e.g., obtained via a finger prick, to identify IgG and/or IgM, if any, produced by an infected individual in response to exposure to the SARS-CoV-2 virus. The sensing element 1206 in turn includes a port 1206b that is configured to receive a mucosal sample, e.g., a sample generated via dissolving a nasal mucosal sample collected via a nasal swab in a diluent (e.g., phosphate buffer solution), or a saliva sample generated by dissolving saliva in a diluent (e.g., phosphate buffer solution) for detecting an IgA antibody generated in response to infection by the SARS-COV-2 virus.

Further in this embodiment, each of the sensing elements 1203 and 1207 includes a graphene layer that is functionalized with the S viral protein. The sensing element 1203 includes a port 1203b configured for receiving a blood sample and the sensing element 1207 includes a portion 1207b configured for receiving a nasal mucosal sample. Thus, the sensing elements 1203 and 1207 can be used to detect IgG/IgM, if present in the collected samples.

In some embodiments, rather than or in addition to separate sensing elements functionalized with S and N viral proteins, one or more sensing elements can be functionalized with both S and N viral proteins.

Further, in this embodiment, the sensor includes sensing elements 1204 and 1208 that include graphene layers functionalized with antibodies exhibiting specific binding to one or two strains of the influenza virus. For example, the sensing element 1204 can be functionalized with antibodies that exhibit specific binding to influenza A virus, such as a monoclonal antibody marketed by Thermo Fisher Scientific under the trade designation (GA2B). And the sensing element 1208 can include a graphene layer functionalized with antibodies exhibiting specific binding to influenza B virus, such as a monoclonal antibody marketed by Invitrogen (B19). The sensing elements 1204 and 1208 includes ports 1204b and 1208b for receiving a sample (a sample obtained via nasal swab) for testing.

In this embodiment, each of the sensing elements includes a connector (e.g., a USB connector) that allows connecting that sensing element to an analyzer according to the present teachings, and detecting an electrical signal generated by the graphene layer in response to exposure to a sample. These connectors are designated as 1201a, 1202a, 1203a, 1204a, 1205a, 1206a, 1207a, and 1208a. The circular form of the sensor allows each of the sensing elements to be readily connected to an analyzer, such as that discussed above, so they can be independently read.

In use, a nasal mucosal sample and a blood sample obtained from an individual can be tested by introducing the nasal swab sample and the blood sample into the different sensing elements configured for detecting the virus itself and the antibodies generated in response to the exposure of the individual to the virus. If the infection is within the period that the virus titer is still detectable, the sensing elements configured to detect the virus can indicate that the infection by the virus has occurred, and if the test is administered about 9-10 days after the onset of the infection when the virus titer has significantly diminished, the presence of the IgM, IgG and/or IgA antibodies can indicate the presence of the infection. In some uses, a person may be tested for immunity after vaccination. The presence of the IgM, IgG and/or IgA antibodies can indicate that immunity has been established after vaccination.

In some embodiments, the above sensor, or a plurality of stand-along sensors, each configured according to the present teachings to detect IgG and/or IgM antibodies can be employed to provide periodic testing of a patient who has tested positive for the SARS-COV-2 virus to monitor the evolution of the infection in that patient.

Further, the above sensor can be employed to test for the influenza virus in a sample collected from the individual. It should be understood that a sensor according to the present teachings can include only one, or a subset, of the above sensing elements.

In some embodiments, at least one of the sensing elements can include a port configured for receiving a fecal sample. This can be particularly helpful for testing infants for infection by the SARS-COV-2 virus. In particular, it has been discovered that in infants under the age of 5, viral shedding can continue for an extended period through feces. As such, infants can be a source of transmission. The testing of infant's fecal samples can be particularly useful in ensuring reducing transmission of the virus. In some embodiments, the fecal sample can be introduced into a buffer, such as those known in the art, and a sample so prepared can be introduced into a sensing element according to the present teachings.

In some embodiments, a sensor according to the present teachings can be configured to detect target nucleotides, such as RNA targets, associated with the SARS-COV-2 virus. In some such sensors, such a functionality can be added to the other functionalities of the sensors discussed above for detecting viral proteins and/or antibodies generated in response to SARS-CoV-2 viral infection. By way of example, and as discussed in more detail below, in some such embodiments, a sensor according to the present teachings can include a plurality of graphene-based sensing elements at least one of which is configured to detect SARS-COV-2 virus via the detection of at least one target RNA of the virus, at least one of which is configured to detect one or more antibodies generated as a result of COVID-19 infection, and at least one graphene-based sensing element that is configured to detect the virus via detection of one or more of its viral proteins, in a manner discussed above.

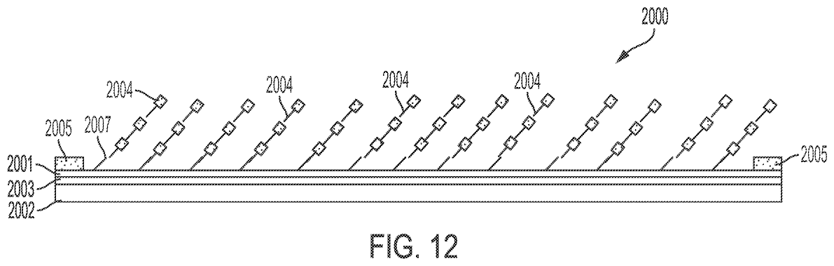

By way of example, FIG. 12 schematically depicts a sensor 2000 according to such an embodiment that includes a graphene layer 2001 that is disposed on an underlying substrate 2002. By way of example, similar to the previous embodiments, the substrate 2002 can be a semiconductor substrate, such as silicon, or a polymeric substrate, such as PDMS. A silicon oxide layer 2003 separates the substrate 2002 from the graphene layer,

In this embodiment, the graphene layer is functionalized with a plurality of oligonucleotides 2004 that exhibit specific binding to one or more RNA targets of SARS-COV-2 virus. By way of example, in some embodiments, the oligonucleotides can be in the form of cDNA corresponding to an RNA target of interest. For example, the cDNA can correspond to the viral envelope (E) gene or a portion thereof. By way of example, such a nucleotide sequence can be as follows: SEQ. ID. 1: ACACTAGCCATCCTTACTGCGCTTCG. In another embodiment, the cDNA probe coupled to the graphene layer for detecting a respective target viral RNA can have the following nucleotide sequence, which corresponds to a portion of the encoding nucleotide sequences for the viral N protein: SEQ. ID. 2: ACCCCGCATTACGTTGGTGGACC. In such cases, the target viral RNA having a complementary sequence can exhibit specific binding to the probe cDNA sequences. In some embodiments, acDNA probe sequence can be attached to the underlying graphene layer via an additional sequence of nucleotides that is attached to the cDNA probe sequence. By way of example, the following nucleotide sequence can be attached to the above cDNA associated with the viral envelope (E) gene: SEQ. ID. 3: GACCCCAAAATCAGCGAAAT. Such additional nucleotide sequences can extend the cDNA probe above the graphene surface, thereby making it more accessible to the complementary viral RNA sequence.

By way of example, the cDNA can be synthesized based on known nucleotide sequence of the target RNA using known synthetic techniques.

In some embodiments, the DNA probe sequence can include additional nucleotides for coupling the DNA probe to the graphene layer in a manner that would allow more facile recognition of the DNA probe by a target RNA sequence. Such additional nucleotides do not necessarily function as probe sequences, but rather can be positioned between the DNA probe sequence and the graphene layer, or a linker connecting the DNA probe sequence to the graphene layer so as to ensure that the probe sequences are sufficiently accessible for specific binding by one or more viral target RNA sequences of interest.

The cDNA can be coupled to the underlying graphene layer using a variety of different methods. For example, in some embodiments, the cDNA can be adsorbed onto the graphene layer via n-n interactions between the nucleobases of the cDNA and the hexagonal cells of graphene. In other embodiments, the cDNA can be covalently bonded to the underlying graphene layer. For example, in this embodiment, the graphene layer can be decorated with —COOH groups 2007 and the cDNA can be covalently attached to such moieties. By way of example, a method of decorating a graphene layer with —COOH groups can be based on 3,4,9,10-perylene tetracarboxylic acid (PTCA), rapid heating and conjugation of acetic acid moieties is known. By way of example, an article entitled “Label-free electrochemical impedance genosensor based on 1-aminopytelene/graphene hybrids,” published in Nanoscale 2013:5:5833-5840, which is herein incorporated by reference in its entirety discloses such a method.

Similar to the previous embodiments, the portions of the graphene layer that are not coupled to cDNA can be passivated via a passivation layer (not shown in FIG. 12), e.g., in a manner discussed above. A plurality of electrodes 2005 deposited on the graphene layer allow measuring an electrical property of the graphene layer, e.g. its electrical resistance, which can change as a result of specific binding of a target viral oligonucleotide, e.g., a target RNA or DNA, to the cDNA probe molecules, thereby allowing the detection of the target viral RNA or DNA In addition, similar to the previous embodiments, the sensor 2000 can include a reference electrode (not shown in this figure) to which a DC-biased AC voltage, such as that schematically depicted in FIG. 10 can be applied to facilitate the detection of an RNA target sequence of interest.

In some embodiments, a sample (e.g., a nasopharyngeal sample) obtained from an individual suspected of having been infected with SARS-COV-2 virus can be processed, for example, in a manner discussed below to release viral RNA and/or DNA from the sample collected from that individual. For example, a lysis/binding buffer containing denaturing agents, such as chaotropic salts and proteinase K, can be employed to release viral RNA and/or DNA In some such embodiments, the buffer can also bind and stabilize the released nucleotides.

In some embodiments, the extracted viral RNA and/or DNA can be amplified, e.g., using isothermal amplification methods, prior to the introduction of the sample onto the sensor. Some examples of such techniques include, without limitation, recombinase polymerase amplification, helicase-dependent amplification, and loop mediated isothermal amplification (LAMP).

The sample can then be introduced onto a sensor according to the present teachings, such as the mechanisms discussed above in connection with the sensors. The specific binding of the RNA target of interest, if present in the sample, with the cDNA probes coupled to the graphene layer can change at least one electrical property of the functionalized graphene layer, e.g., its DC electrical resistance, and thus lead to the detection of the viral RNA

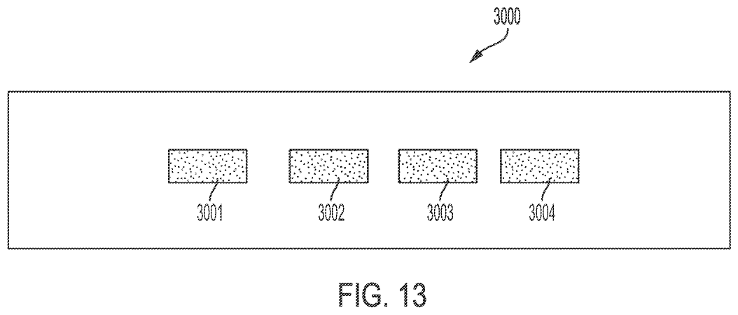

FIG. 13 schematically depicts a sensor 3000 according to another embodiment, which includes an array of graphene-based sensing elements 3001, 3002, 3003, and 3004. In this embodiment, the sensing element 3001 is configured in a manner discussed above to detect SARS-COV-2 virus via interaction between at least one viral protein and an antibody attached to the graphene layer, the sensing element 3002 is configured in a manner discussed above to detect one type of antigen (e.g., IgG and/or IgM) in a sample collected from an infected individual and the sensing element 3003 is configured in a manner discussed above to detect another type of antigen (e.g., IgA) in a sample collected from an infected individual. A sensor according to the present teachings provides a fast, cost-effective and easy-to-use tool that can be employed to detect SARS-COV-2 in a biological sample, e.g., a fecal sample.

The following Example provides a method of producing monoclonal antibodies that exhibit specific binding to at least one epitope of SARS-COV-2 virus.

### EXAMPLE 1

### Generation of Mouse Monoclonal Antibodies

Fifty micrograms of recombinant SARS-COV-2 viral protein suspended in phosphate buffered saline (PBS; GIBCO, Grand Island, NY) and emulsified with an equal volume of complete Freund's adjuvant (Sigma Chemical Co., St. Louis, MO). Mice are immunized by injection of the emulsion at three subcutaneous sites and one intraperitoneal (i.p.) site. Fourteen days after the initial immunization, the mice are given a booster immunization i.p. with twenty-five micrograms of recombinant SARS-COV-2 viral protein suspended in PBS and emulsified with an equal volume of incomplete Freund's adjuvant. A second booster of 25 ug recombinant SARS-COV-2 viral protein in PBS was given after another 14 days. Ten days later, a small amount of blood was collected and the serum activity against of the recombinant SARS-COV-2 viral protein is assessed by titer using indirect enzyme-linked immunosorbent assay (ELISA) with recombinant SARS-COV-2 viral protein or an irrelevant protein (negative control) bound to the plates. The mouse with the best titer is rested for 3 weeks after the last immunization and then boosted by intravenous injection of 25 ug recombinant SARS-COV-2 viral protein in PBS. Three days later the mouse is euthanized and the spleen and lymph nodes are collected and made into a cell suspension, then washed with Dulbecco's modified Eagle's medium (DMEM). The spleen/lymph node cells are counted and mixed with SP 2/0 myeloma cells (ATCC No. CRL8-006, Rockville, MD) that are incapable of secreting either heavy or light chain immunoglobulin chains (Kearney et al., 1979) using a spleen: myeloma ratio of 2:1. Cells are fused together using polyethylene glycol 1450 (ATCC) into eight 96-well tissue culture plates in hypoxanthine-aminopterin-thymidine (HAT) selection medium according to standard procedures (Kohler and Milstein, 1975).

Between 10 and 21 days after fusion, hybridoma colonies become visible and culture supernatants are harvested then screened by ELISA using high-protein binding 96-well enzyme immunoassay (EIA) plates (Costar/Corning, Inc. Corning, NY) coated with 50 μl/well of a 2 μg/ml solution (0.1 μg/well) ofrecombinant SARS-COV-2 viral protein or an irrelevant protein negative control and incubated overnight at 4° C. The excess solution is aspirated and the plates are washed with PBS/0.05% Tween-20 (three times), then blocked with 1% bovine serum albumin (BSA, fraction V, Sigma Chemical Co., MO) for 1 hr at room temperature (RT) to reduce non-specific binding. The BSA solution was removed and 50 ul/well of hybridoma supernatant from each fusion plate well are added. The plates are then incubated for 45 min. at 37° C. and washed three times with PBS/0.05% Tween-20. Horseradish peroxidase (HRP)-conjugated goat anti-mouse IgG F(ab)2 (H&L) (Jackson Research Laboratories, Inc., West Grove, PA) is diluted 1:4000 in 19% BSA/PBS then added to each well. The plates are then incubated for 45 min. at 37° C. After washing in PBS, 50 ul/well of 3,3′,5,5′-Tetramethylbenzidine (TMB) substrate solution (ThermoFisher) was added to work with the HRP and produce a blue color. After a few minutes the reaction is inhibited using 0.16M sulfuric acid solution (ThermoFisher), turning the blue color into a yellow color. The intensity of the yellow color of positive wells at 450 nm is assessed using a Spectramax190 microtiter plate reader (Molecular Devices Corp., Sunnyvale, CA). Further details regarding the production of antibodies can be found in Kearney, JF; Radbruch, A; Liesegang, B; Rajewsky, K. 1979. A new mouse myeloma cell line that has lost immunoglobulin expression but permits the construction of antibody-secreting hybrid cell lines. J Immunol 123:1548-1550. and Kobler, G; Milstein, C. 1975. Continuous cultures of fused cells secreting antibody of predefined specificity. Nature 256:495-497, which are herein incorporated by reference in their entirety.

### EXAMPLE 2

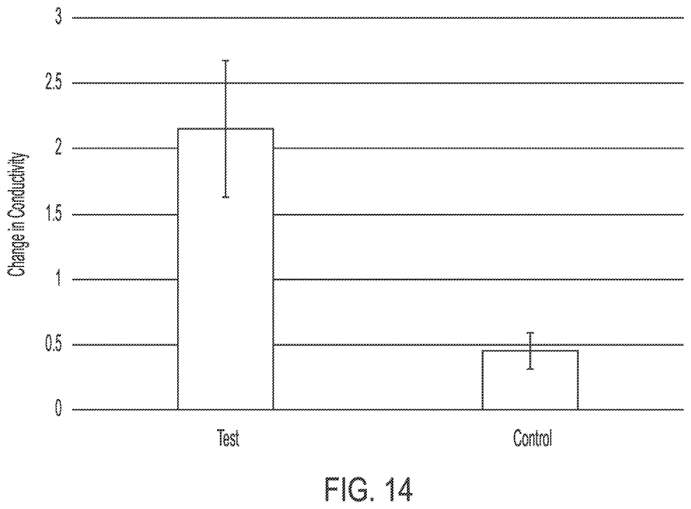

A prototype sensor according to the present teachings was fabricated as described above and tested using samples containing spike proteins that represent SARS-COV-2 virus. The graphene layer of the sensor was functionalized with anti-spike protein antibodies. Further, the unfunctionalized portions of the graphene layer were passivated using polyethylene glycol (PEG) and ethanolamine. A control sensor was also fabricated, in which the graphene layer was functionalized with an isotype control antibodies. Both sensors were loaded with samples including the spike protein of the SARS-COV-2 virus, and the electric conductivity was measured. As shown in Table 1 and FIG. 14, the prototype sensor exhibited a higher change in conductivity (i.e., a percentage change in conductivity or electron mobility between a buffer containing no spike proteins and a sample containing spike proteins) in response to interaction with the spike proteins, which was statistically higher than that exhibited by the control sensor.

TABLE 1

| | |
|---|---|
| Test | Control |

Antibody

| | |
|---|---|
| Anti-spike | Isotype |

Protein

| | | Spike protein | Irrev. Protein |
|---|---|---|---|
| Change in | Chip 1 | 1.561763607 | 0.295613102 |
| Conductivity | Chip 2 | 2.316691922 | 0.632826175 |
| | Chip 3 | 2.572200291 | 0.46214932 |
| | Chip 4 | | 0.417339312 |

| | | |
|---|---|---|
| Average | 2.150218607 | 0.451981977 |
| Standard Deviation | 0.525386141 | 0.139593861 |

In various embodiments, one or more of disclosed modules are implemented via one or more computer programs for performing the functionality of the corresponding modules, or via computer processors executing those programs. In some embodiments, one or more of the disclosed modules are implemented via one or more hardware modules executing firmware for performing the functionality of the corresponding modules. In various embodiments, one or more of the disclosed modules include storage media for storing data used by the module, or software or firmware programs executed by the module. In various embodiments, one or more of the disclosed modules or disclosed storage media are internal or external to the disclosed systems. In some embodiments, one or more of the disclosed modules or storage media are implemented via a computing “cloud”, to which the disclosed system connects via a network connection and accordingly uses the external module or storage medium. In some embodiments, the disclosed storage media for storing information include non-transitory computer-readable media, such as a CD-ROM, a computer storage, e.g., a hard disk, or a flash memory. Further, in various embodiments, one or more of the storage media are non-transitory computer-readable media that store data or computer programs executed by various modules, or implement various techniques or flow charts disclosed herein.

The above detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

1-28. (canceled)

29. A sensor for detecting at least one pathogen, comprising:
a first sensing element, and a second sensing element, wherein the first sensing element includes:
a first functionalized graphene layer including:
a first graphene layer; and
a first binding agent coupled to the first graphene layer, wherein the first binding agent binds to an epitope of a pathogen; and
first electrical conductors electrically coupled to the first functionalized graphene layer and configured to measure a property of the first functionalized graphene layer,
wherein the second sensing element includes:
a second functionalized graphene layer including:
a second graphene layer;
a second binding agent coupled to the second layer, wherein the second binding agent binds to an antibody produced in a subject in response to infection by the pathogen; and
second electrical conductors electrically coupled to the second functionalized graphene layer and configured to measure an electrical property of the second functionalized graphene layer.

30. The sensor of claim 29, wherein the at least one pathogen is a coronavirus, chlamydia or influenza.

31. The sensor of claim 30, wherein the coronavirus is SARS-COV-2.

32. The sensor of claim 29, wherein the first functionalized graphene layer includes a first linker with a first end and a second end, wherein the first end of the first linker is coupled to the first graphene layer via a π-πbond and the second end of the first linker is coupled to the first binding agent, and the second functionalized graphene layer includes a second linker with a first end, wherein the first end of the second linker is coupled to the second graphene layer via a π-πbond and the second end of the second linker is coupled to the second binding agent.

33. The sensor of claim 29, wherein the first binding agent includes an antibody.

34. The sensor of claim 29, wherein the second binding agent includes protein of the pathogen.

35. The sensor of claim 29, further comprising a third sensing element including:
a third functionalized graphene layer including:
a third graphene layer;
a third binding agent coupled to the third graphene layer, wherein the third binding agent includes an antibody that binds to an epitope of a first strain of influenza; and
third electrical conductors electrically coupled to the third functionalized graphene layer and configured to measure an electrical property of the third functionalized graphene layer.

36. The sensor of claim 35, further comprising a fourth sensing element sensing element including:
a fourth functionalized graphene layer including:
a fourth graphene layer;
a fourth binding agent coupled to the fourth graphene layer, wherein the fourth binding agent includes an antibody that binds to an epitope of a different strain of influenza; and
fourth electrical conductors electrically coupled to the fourth functionalized graphene layer and configured to measure an electrical property of the fourth functionalized graphene layer.
